(19) United States
(12) Patent Application Publication
(10) Pub. No.: US 2024/0247065 A1
(43) Pub. Date: Jul. 25, 2024

(54) METHODS OF TREATMENT OF CANCER PATIENTS HAVING ALTERED SETD2 BIOMARKER WITH A PD-1 ANTAGONIST

(71) Applicant: MERCK SHARP & DOHME LLC, RAHWAY, NJ (US)

(72) Inventors: Razvan CRISTESCU, Newton, MA (US); Elisha DETTMAN, Landsdale, PA (US); Yongjin LI, Newton, MA (US); Karla Gabriela RODRIGUEZ-LOPEZ, Westfield, NJ (US); Michael NEBOZHYN, Colmar, PA (US); Rodolfo Fleury PERINI, Wynnewood, PA (US); Raluca Andreia PREDOIU, Penn Valley, PA (US); Yiwei ZHANG, Katy, TX (US)

(73) Assignee: MERCK SHARP & DOHME LLC, RAHWAY, NJ (US)

(21) Appl. No.: 18/561,331

(22) PCT Filed: May 16, 2022
(86) PCT No.: PCT/US2022/029372

§ 371 Date: Nov. 16, 2023

Related U.S. Application Data
(60) Provisional application No. 63/190,494, filed on May 19, 2021.

(51) Int. Cl. C07K 16/28; A61K 39/00; C12Q 1/6886

(52) U.S. Cl. CPC C07K 16/2818; C12Q 1/6886; A61K 2039/505; A61K 2039/545; C07K 2317/24; C07K 2317/565; C12Q 2600/106; C12Q 2600/156

(57) ABSTRACT

The present disclosure describes methods of treatment of cancer in patients with altered activity or amount of a SETD2 biomarker with a treatment regimen comprising an antagonist of Programmed Death 1 receptor (PD-1).

### CROSS-REFERENCE TO RELATED APPLICATIONS

This application claims the benefit of U.S. Provisional Application No. 63/190,494, filed on May 19, 2021, the contents of which are hereby incorporated by reference in their entirety.

### FIELD OF THE INVENTION

The present disclosure describes methods of treatment of cancer in patients with altered activity or amount of a SETD2 biomarker with an antagonist of Programmed Death 1 receptor (PD-1).

### REFERENCE TO SEQUENCE LISTING SUBMITTED ELECTRONICALLY

The sequence listing of the present application is submitted electronically via EFS-Web as an ASCII formatted sequence listing with a file name “25249WOPCT-SEQLIST-26APR2022.TXT”, creation date of Apr. 26, 2022, and a size of 15.2 KB. This sequence listing submitted via EFS-Web is part of the specification and is herein incorporated by reference in its entirety.

### BACKGROUND OF THE INVENTION

PD-1 is recognized as an important molecule in immune regulation and the maintenance of peripheral tolerance. PD-1 is moderately expressed on naive T, B and NKT cells and up-regulated by T/B cell receptor signaling on lymphocytes, monocytes and myeloid cells (Sharpe, Arlene H et al., The function of programmed cell death 1 and its ligands in regulating autoimmunity and infection. Nature Immunology (2007): 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong, Haidong et al., Tumor-associated B7-H1 promotes T-cell apoptosis: a potential mechanism of immune evasion. Nat Med. 2002 August: 8(8):793-800; Yang, Wanhua et al., PD-1 interaction contributes to the functional suppression of T-cell responses to human uveal melanoma cells in vitro. Invest Ophthalmol Vis Sci. 2008 June: 49(6 (2008): 49: 2518-2525; Ghebeh, Hazem et al., The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198: Hamanishi, Junzo et al., Programmed cell death 1 ligand 1 and tumor-infiltrating CD8+ T lymphocytes are prognostic factors of human ovarian cancer. Proc. Natl. Acad. Sci, USA (2007): 104: 3360-3365: Thompson, R Houston, and Eugene D Kwon, Significance of B7-H1 overexpression in kidney cancer. Clinical genitourin Cancer (2006): 5: 206-211: Nomi, Takeo et al., Clinical significance and therapeutic potential of the programmed death-1 ligand/programmed death-1 pathway in human pancreatic cancer. Clinical Cancer Research (2007): 13:2151-2157: Ohigashi, Yuichiro et al., Clinical significance of programmed death-1 ligand-1 and programmed death-1 ligand 2 expression in human esophageal cancer. Clin. Cancer Research (2005): 11: 2947-2953; Inman, Brant A et al., PD-L1 (B7-H1) expression by urothelial carcinoma of the bladder and BCG-induced granulomata: associations with localized stage progression. Cancer (2007): 109: 1499-1505: Shimauchi, Takatoshi et al., Augmented expression of programmed death-1 in both neoplasmatic and nonneoplastic CD4+ T-cells in adult T-cell Leukemia/Lymphoma. Int. J. Cancer (2007): 121:2585-2590; Gao, Qiang et al., Overexpression of PD-L1 significantly associates with tumor aggressiveness and postoperative recurrence in human hepatocellular carcinoma. Clinical Cancer Research (2009) 15: 971-979; Nakanishi, Juro et al., Overexpression of B7-H1 (PD-L1) significantly associates with tumor grade and postoperative prognosis in human urothelial cancers. Cancer Immunol Immunother. (2007) 56: 1173-1182: Hino et al., Tumor cell expression of programmed cell death-1 is a prognostic factor for malignant melanoma. Cancer (2010): 00: 1-9). Similarly, PD-1 expression on tumor infiltrating lymphocytes was found to mark dysfunctional T cells in breast cancer and melanoma (Ghebeh, Hazem et al., Foxp3+ tregs and B7-H1+/PD-1+ T lymphocytes co-infiltrate the tumor tissues of high-risk breast cancer patients: implication for immunotherapy. BMC Cancer. 2008 February 23:8:57: Ahmadzadeh, Mojgan et al., Tumor antigen-specific CD8 T cells infiltrating the tumor express high levels of PD-1 and are functionally impaired. Blood (2009) 114: 1537-1544) and to correlate with poor prognosis in renal cancer (Thompson, R Houston et al., PD-1 is expressed by tumor infiltrating cells and is associated with poor outcome for patients with renal carcinoma. Clinical Cancer Research (2007) 15: 1757-1761). Thus, it has been proposed that PD-L1-expressing tumor cells interact with PD-1-expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab (KEYTRUDA®, Merck & Co., Inc., Rahway, NJ, USA) is a potent humanized immunoglobulin G4 (IgG4) mAb with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications.

Renal cell carcinoma (RCC) is characterized by frequent alterations in von Hippel Lindau protein (VHL), a tumor suppressor that regulates the transcription factor hypoxia inducible factor (HIF) central to controlling angiogenesis. Alterations in SWItch/Sucrose Non-Fermentable (SWI/SNF) chromatin remodeling complex are also common. SWI/SNF subunits commonly mutated in clear cell RCC (ccRCC) include polybromo 1 (PBRM1), AT-Rich Interaction Domain IA (ARID1A), and Transcription activator BRG1 (SMARCA4). Other commonly mutated genes included the histone deubiquitinase BRCA1 Associated Protein 1 (BAP1), and the histone methyltransferase SET domain containing 2 (SETD2). The genes encoding VHL, PBRM1, BAP1, and SETD2 are all clustered in the small arm of chromosome 3 (chr3p), and arm-level deletions of chr3p are exceedingly common in ccRCC (>90% of samples: TCGA (2013) Nature 499:43-49).

SET-Domain Containing 2 (SETD2) is an active tumor suppressor in breast cancer and leukemia that lies on chromosome 3p, an area that is frequently deleted in ccRCC. Its mutation occurs in up to 16% of sporadic ccRCC cases. SETD2 mediates chromatin remodeling by regulating the transcription of genes and modifying histones, and it plays a significant role in DNA damage repair. SETD2 encodes a methyltransferase and is responsible for trimethylation of lysine-36 of histone H3, generating its canonical histone modification product, H3K36me3 (Santos, Victor Espinheira et al., Prognostic impact of loss of SETD2 in clear cell renal cell carcinoma patients. Clinical Genitourinary Cancer, 19, 4 (2021): 339-345; Edmunds, John W et al., Dynamic histone H3 methylation during gene induction: HYPB/Setd2 mediates all H3K36 trimethylation. The EMBO journal vol. 27, 2 (2008): 406-20).

There have been recent advances in the treatment of first line (1L) advanced renal cell carcinoma (RCC) combining immunomodulators and/or VEGF receptor tyrosine kinase inhibitors (VEGFR-TKI(s)), and multiple agents are also now available for the treatment of patients with second line (2L) RCC. However, existing data shows that few patients experience complete response (CR) with these agents and nearly all experience progression of their cancer. Although these significant advances have led to a change in the treatment paradigm for RCC patients, there remains an unmet need to improve outcomes for both 1L and 2L+ advanced RCC populations.

### SUMMARY OF THE INVENTION

The invention provides a method of treating a patient diagnosed with cancer who has been identified as having an altered SETD2 biomarker comprising administering a PD-1 antagonist to the patient. In some embodiments, the altered SETD2 biomarker is an altered activity or altered amount of a SETD2 biomarker. In one embodiment, the altered activity or amount is reduced activity or amount of the SETD2 biomarker. In another embodiment, the altered activity is a SETD2 biomarker loss-of-function mutation. In one embodiment, the altered SETD2 biomarker is a reduced copy number of SETD2 DNA sequences in a cell. In another embodiment, the altered SETD2 biomarker is an altered structure of a SETD2 biomarker. The invention also provides methods of identifying the likelihood of a cancer in a subject to be responsive to treatment with a PD-1 antagonist by identifying a somatic mutation in the SETD2 nucleic acid in a sample from the subject. In one embodiment, the cancer is renal cell carcinoma. In one embodiment, the PD-1 antagonist is an anti-PD-1 antibody that blocks the binding of PD-1 to PD-L1 and/or PD-L2.

Also provided herein is a method for measuring the amount and/or activity of a SETD2 biomarker in a sample from a subject. The invention further relates to identification of a patient who has altered activity of a SETD2 biomarker, e.g., has reduced activity of a SETD2 biomarker.

### DETAILED DESCRIPTION

Abbreviations. Throughout the detailed description and examples of the invention the following abbreviations will be used:
BOR Best overall response
BID One dose twice daily
BICR Blinded Independent Central Radiology
CBR Clinical Benefit Rate
CDR Complementarity determining region
CHO Chinese hamster ovary
CR Complete Response
DCR Disease Control Rate
DFS Disease free survival
DLT Dose limiting toxicity
DOR Duration of Response
DSDR Durable Stable Disease Rate
FFPE Formalin-fixed, paraffin-embedded
FR Framework region
IgG Immunoglobulin G
IHC Immunohistochemistry or immunohistochemical
irRC Immune related response criteria
IV Intravenous
MTD Maximum tolerated dose
NCBI National Center for Biotechnology Information
NCI National Cancer Institute
ORR Objective response rate
OS Overall survival
PD Progressive disease
PD-1 Programmed Death 1
PD-L1 Programmed Cell Death 1 Ligand 1
PD-L2 Programmed Cell Death 1 Ligand 2
PFS Progression free survival
PR Partial response
Q2W One dose every two weeks
Q3W One dose every three weeks
Q4W One dose every four weeks
Q6W One dose every six weeks
QD One dose per day
RECIST Response Evaluation Criteria in Solid Tumors
SD Stable disease
TPI Toxicity Probability Interval
VH Immunoglobulin heavy chain variable region
VK Immunoglobulin kappa light chain variable region

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a.” “an,” and “the,” include their corresponding plural references unless the context clearly dictates otherwise.

“About” when used to modify a numerically defined parameter (e.g., the gene signature score for a gene signature discussed herein, or the dosage of a PD-1 antagonist, or the length of treatment time with a PD-1 antagonist, or the amount of time between treatments with a PD-1 antagonist) means that the parameter may vary by as much as 10% above or below the stated numerical value for that parameter. For example, a gene signature consisting of about 10 genes may have between 9 and 11 genes. Similarly, a reference gene signature score of about 2.462 includes scores of and any score between 2.2158 and 2.708. In certain embodiments, “about” can mean a variation of ±0.1%, ±0.5%, ±1%, ±2%, ±3%, ±4%, ±5%, ±6%, ±7%, ±8%, +9% or ±10%. When referring to the amount of time between administrations in a therapeutic treatment regimen (i.e., amount of time between administrations of the PD-1 antagonist, e.g. “about 6 weeks,” which is used interchangeably herein with “approximately every six weeks”), “about” refers to the stated time+a variation that can occur due to patient/clinician scheduling and availability around the 6-week target date. For example, “about 6 weeks” can refer to 6 weeks±5 days, 6 weeks±4 days, 6 weeks±3 days, 6 weeks±2 days or 6 weeks #1 day, or may refer to 5 weeks, 2 days through 6 weeks, 5 days.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “subject” includes any organism, preferably an animal, more preferably a mammal (e.g., rat, mouse, dog, cat, rabbit) and most preferably a human.

The term “altered activity” of a biomarker refers to an activity of the biomarker which may be increased or decreased in a disease state, e.g., in a cancer sample, as compared to the activity of the biomarker in a normal, control sample (e.g. a non-cancerous tissues sample). Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors. The term “altered structure” of a biomarker refers to the presence of mutations or allelic variants within a biomarker nucleic acid or protein, e.g., mutations that affect expression or activity of the biomarker nucleic acid or protein, as compared to the normal or wild-type gene or protein. For example, mutations include, but are not limited to nucleotide or amino acid substitutions, deletions, or additions. Mutations may be present in the coding or non-coding region of the biomarker nucleic acid. An altered activity of SETD2 protein can be determined by detecting the methylation status of its substrate lysine-36 of histone H3. An antibody which specifically binds to the methylated lysine at amino acid position 36 of histone 3 can be used for immunostaining to determine the activity of SETD2 in a cancerous sample of a patient.

The term “altered amount” or “altered level” refers to increased or decreased copy number (e.g., germline and/or somatic) of a biomarker nucleic acid, e.g., a gene or DNA, or an increased or decreased expression level of a gene (i.e. transcription or translation) that may be detectable in a cancer sample, as compared to the expression level or copy number of the biomarker nucleic acid in a control sample. The term “altered amount” of a biomarker also includes an increased or decreased protein level of a biomarker protein that may be present in a sample, e.g., a cancer sample, as compared to the corresponding protein level in a normal, control sample.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). An SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such an SNP may alter the coding sequence of the gene and therefore specify another amino acid (a “missense” SNP) or an SNP may introduce a stop codon (a “nonsense” SNP). When an SNP does not alter the amino acid sequence of a protein, the SNP is called “silent.” SNP's may also occur in noncoding regions of the nucleotide sequence. This may result in defective protein expression, e.g., as a result of alternative splicing, or it may have no effect on the function of the protein.

As used herein, the term “gene” refers to nucleic acid molecules comprising an open reading frame encoding a polypeptide corresponding to a biomarker of the present invention.

“SETD2 biomarker” as used herein refers to SETD2 protein (amino acid encoded by cDNA, Genbank accession No. NM_014159.6), mRNA, genomic DNA (in genome GRCh37/hg19 chr3:47,057,898-47,205,467) or cDNA (Genbank accession No. NM_014159.6).

An “altered SETD2 biomarker,” as used herein, refers to an altered amount or altered level of a SETD2 biomarker, altered activity of a SETD2 biomarker, or altered structure of a SETD2 biomarker (e.g. biomarker sequence alteration/mutation, allelic variant).

“Somatic mutation in the SETD2 nucleic acid” as used herein refers to genetic alteration acquired by a cell that can be passed to the progeny of the mutated cell in the course of cell division. Somatic mutations differ from germ line mutations, which are inherited genetic alterations that occur in the germ cells. The somatic mutations include but are not limited to single-base nucleotide substitutions, multi-base nucleotide substitutions, insertion mutations, deletion mutations, frameshift mutations, missense mutations, nonsense mutations, splice-site mutations, and combinations thereof.

The term “SETD2 biomarker loss-of-function mutation” or “loss-of-function mutation” refers to any mutation in a SETD2 nucleic acid or protein that results in reduced or eliminated SETD2 protein amounts and/or function. For example, nucleic acid mutations include single-base substitutions, multi-base substitutions, insertion mutations, deletion mutations, frameshift mutations, missense mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. Such mutations reduce or eliminate SETD2 protein amounts and/or function by eliminating proper coding sequences required for proper SETD2 protein translation and/or coding for SETD2 proteins that are nonfunctional or have reduced function (e.g., deletion of enzymatic and/or structural domains, reduction in protein stability, alteration of sub-cellular localization, and the like). Such mutations are well-known in the art. In some embodiments, the mutation is a “nonsynonymous mutation,” meaning that the mutation alters the amino acid sequence of SETD2. In addition, a representative list describing a wide variety of loss-of-function mutations is described in Table 3 of the Examples.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2. FR3, CDR3 and FR4. The assignment of amino acids to each domain is, generally, in accordance with the definitions of Sequences of Proteins of Immunological Interest, Kabat, et al.: National Institutes of Health, Bethesda, Md.; 5^{th} ed.: NIH Publ. No. 91-3242 (1991); Kabat (1978) Adv. Prot. Chem. 32:1-75: Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of an antibody, i.e. antibody fragments that retain the ability to bind specifically to the antigen bound by the full-length antibody, e.g. fragments that retain one or more CDR regions, e.g. all six CDRs. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response,” when referring to a cancer patient treated with a treatment regimen, such as a therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival.

Positive therapeutic effects in cancer can be measured in a number of ways (See, W. A. Weber, J. Null. Med. 50:1S-10S (2009); Eisenhauer et al., supra). In some embodiments, an anti-tumor response to a therapy described herein is assessed using RECIST 1.1 criteria, bidimensional irRC or unidimensional irRC. In some embodiments, an anti-tumor response is any of SD, PR, CR, PFS, or DFS.

“Bidimensional irRC” refers to the set of criteria described in Wolchok J D, et al. Guidelines for the evaluation of immune therapy activity in solid tumors: immune-related response criteria. Clin Cancer Res. 2009:15(23):7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response. Classes of biotherapeutic agents include, but are not limited to, antibodies to PD-1, LAG3, VEGF, EGFR, Her2/neu, other growth factor receptors, CD20, CD40, CD-40L, CTLA-4, OX-40, 4-1BB, and ICOS.

“CBR” or “Clinical Benefit Rate” means CR+PR+durable SD.

“CDR” or “CDRs” as used herein means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, luteinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in Al-Lazikani et al., JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Combination therapy” or “in combination” refers to two or more biotherapeutic and chemotherapeutic agents administered as a part of a treatment regimen.

“In sequence” refers to two or more treatment regimens administered sequentially in any order.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“DCR” or “Disease Control Rate” means CR+PR+SD.

“DSDR” or “Durable Stable Disease Rate” means SD for ≥23 weeks.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example, See also Presta (2005) J. Allergy Clin. Immunol. 116:731.

“Non-responder patient”, when referring to a specific anti-tumor response to treatment with a therapy described herein, means the patient did not exhibit the anti-tumor response.

“ORR” or “objective response rate” refers in some embodiments to CR+PR, and ORR(week 24) refers to CR and PR measured using irRECIST in each patient in a cohort after 24 weeks of anti-cancer treatment.

“Patient” or “subject” refers to any single subject for which therapy is desired or that is participating in a clinical trial, epidemiological study or used as a control, including humans and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or Natural Killer T cell) and in specific embodiments also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and in specific embodiments blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

“Pembrolizumab” (formerly known as MK-3475, SCH 900475 and lambrolizumab) alternatively referred to herein as “pembro.” is a humanized IgG4 mAb with the structure described in WHO Drug Information, Vol. 27, No. 2, pages 161-162 (2013) and which comprises the heavy and light chain amino acid sequences and CDRs described in Table 2. Pembrolizumab has been approved by the U.S. FDA as described in the Prescribing Information for KEYTRUDA™ (Merck & Co., Inc., Rahway, NJ, USA: initial U.S. approval 2014, updated March 2021).

As used herein, a “pembrolizumab variant” means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“RECIST 1.1 Response Criteria” as used herein means the definitions set forth in Eisenhauer et al., E. A, et al., Eur. J Cancer 45:228-247 (2009) for target lesions or nontarget lesions, as appropriate based on the context in which response is being measured.

“Responder patient” when referring to a specific anti-tumor response to treatment with a therapy described herein, means the patient exhibited the anti-tumor response.

“Sample” when referring to a tumor or any other biological material referenced herein, means a tissue sample that has been removed from the subject's tumor: thus, the testing methods described herein are not performed in or on the subject (although the methods of treatment of the invention clearly include treating the subject).

“Sustained response” means a sustained therapeutic effect after cessation of treatment with a therapeutic agent, or a therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” cancer as used herein means to administer therapy to a subject having cancer, or diagnosed with cancer, to achieve at least one positive therapeutic effect, such as for example, reduced number of cancer cells, reduced tumor size, reduced rate of cancer cell infiltration into peripheral organs, or reduced rate of tumor metastasis or tumor growth.

Positive therapeutic effects in cancer can be measured in a number of ways (See, W. A. Weber, J. Nucl. Med. 50:1S-10S (2009)). For example, with respect to tumor growth inhibition, according to NCI standards, a ratio between the tumor volume in the treated group and in the untreated control group (T/C)≤42% is the minimum level of anti-tumor activity. A T/C<10% is considered a high anti-tumor activity level, with T/C (%)=Median tumor volume of the treated/Median tumor volume of the control ×100. In some embodiments, response to a therapy described herein is assessed using RECIST 1.1 criteria or irRC (bidimensional or unidimensional) and the treatment achieved by a therapy of the invention is any of PR, CR, OR, PFS, DFS and OS.

PFS, also referred to as “Time to Tumor Progression” indicates the length of time during and after treatment that the cancer does not grow; and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a therapy of the invention is any of PR, CR, PFS, DFS, OR and OS that is assessed using RECIST 1.1 response criteria. The treatment regimen for a therapy of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The terms “dosing protocol” and “dosing regimen” are used interchangeably to refer to the dose and timing of administration of one or more therapeutic agents.

The term “treatment regimen” refers to the administration of one or more therapeutic agents in a treatment.

The term “therapeutically effective amount” refers to non-toxic and sufficient amount of the therapeutic agent to provide an anti-tumor response or to bring about a positive therapeutic effect in a patient. The amount needed to elicit the therapeutic response can be determined based on the age, health, size and sex of the subject. Optimal amounts can also be determined based on monitoring of the subject's response to treatment.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow: Tumor burden can be determined by a variety of methods known in the art, such as, by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC” refers to the set of criteria described in Nishino M, Giobbie-Hurder A, Gargano M, Suda M, Ramaiya N H, Hodi F S. Developing a Common Language for Tumor Response to Immunotherapy: Immune-related Response Criteria using Unidimensional measurements. Clin Cancer Res. 2013: 19(14):3936-3943). These criteria utilize the longest diameter (cm) of each lesion.

“Variable regions” or “V region” as used herein means the segment of IgG chains which is variable in sequence between different antibodies. Typically, it extends to Kabat residue 109 in the light chain and 113 in the heavy chain.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin lymphoma, non-Hodgkin lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. In some embodiments, cancers that may be treated in accordance with the present invention include those characterized by elevated expression of one or both of PD-L1 and PD-L2 in tested tissue samples.

### PD-1 Antagonists

PD-1 antagonists useful in the treatment method, medicaments and uses of the present invention include a monoclonal antibody (mAb), or antigen binding fragment thereof, that specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the treatment method, medicaments and uses of the present invention, are described in U.S. patent nos. U.S. Pat. No. 7,488,802. U.S. Pat. Nos. 7,521,051, 8,008,449, 8,354,509, and 8,168,757, and International application publn. nos. WO2004/004771, WO2004/072286, WO2004/056875, US2011/0271358, and WO 2008/156712. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information. Vol. 27, No. 2, pages 161-162 (2013) and that comprises the heavy and light chain amino acid sequences shown in Table 2: nivolumab (BMS-936558), a human IgG4 mAb with the structure described in WHO Drug Information. Vol. 27, No. 1, pages 68-69 (2013) and that comprises the heavy and light chain amino acid sequences shown in Table 2: the humanized antibodies h409A11, h409A16 and h409A17, which are described in WO2008/156712, and AMP-514, which is being developed by MedImmune; cemiplimab; camrelizumab; sintilimab; tislelizumab; and toripalimab. Additional anti-PD-1 antibodies contemplated for use herein include MEDI0680 (U.S. Pat. No. 8,609,089), BGB-A317 (U.S. Patent publ, no. 2015/0079109). INCSHR1210 (SHR-1210) (PCT International application publ, no. WO2015/085847), REGN-2810 (PCT International application publ, no. WO2015/112800). PDR001 (PCT International application publ, no. WO2015/112900). TSR-042 (ANB011) (PCT International application publ, no. WO2014/179664) and STI-1110 (PCT International application publ, no. WO2014/194302).

Examples of mAbs that bind to human PD-L1, and useful in the treatment method, medicaments and uses of the present invention, are described in U.S. Pat. No. 8,383,796. Specific anti-human PD-L1 mAbs useful as the PD-1 antagonist in the treatment method, medicaments and uses of the present invention include BMS-936559. MEDI4736, and MSB0010718C.

In some embodiments, the PD-1 antagonist is pembrolizumab (KEYTRUDA™, Merck & Co., Inc., Kenilworth, NJ, USA), nivolumab (OPDIVO™, Bristol-Myers Squibb Company. Princeton, NJ, USA), atezolizumab (TECENTRIQ™, Genentech, San Francisco, CA, USA), durvalumab (IMFINZI™. AstraZeneca Pharmaceuticals LP, Wilmington, DE), cemiplimab (LIBTAYO™, Regeneron Pharmaceuticals, Tarrytown, NY, USA) or avelumab (BAVENCIO™, Merck KGaA, Darmstadt, Germany). In other embodiments, the PD-1 antagonist is pidilizumab (U.S. Pat. No. 7,332,582). AMP-514 (MedImmune LLC. Gaithersburg, MD, USA), PDR001 (U.S. Pat. No. 9,683,048). BGB-A317 (U.S. Pat. No. 8,735,553), and MGA012 (MacroGenics, Rockville, MD).

Other PD-1 antagonists useful in the treatment methods, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in PCT International application public, nos. WO2010/027827 and WO2011/066342. Specific fusion proteins useful as the PD-1 antagonist in the treatment methods, medicaments and uses of the present invention include AMP-224 (also known as B7-DCIg), which is a PD-L2-FC fusion protein and binds to human PD-1.

In one embodiment, the PD-1 antagonist useful in the methods of the invention is an anti-PD-1 antibody that blocks the binding of PD-1 to PD-L1 and PD-L2. In some embodiments of the treatment methods, medicaments and uses of the present invention, the PD-1 antagonist is a monoclonal antibody, or antigen binding fragment thereof, that comprises: (a) a light chain variable region comprising light chain CDR1, CDR2 and CDR3 of SEQ ID NOs: 1, 2 and 3, respectively and (b) a heavy chain variable region comprising heavy chain CDR1. CDR2 and CDR3 of SEQ ID NOs: 6, 7 and 8, respectively.

In other embodiments of the treatment methods, medicaments and uses of the present invention, the PD-1 antagonist is a monoclonal antibody, or antigen binding fragment thereof, that specifically binds to human PD-1 and comprises (a) a heavy chain variable region comprising SEQ ID NO:9 or a variant thereof, and (b) a light chain variable region comprising SEQ ID NO:4 or a variant thereof. A variant of a heavy chain variable region sequence is identical to the reference sequence except having up to six conservative amino acid substitutions in the framework region (i.e., outside of the CDRs). A variant of a light chain variable region sequence is identical to the reference sequence except having up to three conservative amino acid substitutions in the framework region (i.e., outside of the CDRs).

In another embodiment of the treatment methods, medicaments and uses of the present invention, the PD-1 antagonist is a monoclonal antibody that specifically binds to human PD-1 and comprises (a) a heavy chain comprising SEQ ID NO: 10 and (b) a light chain comprising SEQ ID NO:5. In one embodiment, the PD-1 antagonist is an anti-PD-1 antibody that comprises two heavy chains and two light chains, and wherein the heavy and light chains comprise the amino acid sequences in SEQ ID NO: 10 and SEQ ID NO:5, respectively.

In yet another embodiment of the treatment methods, medicaments and uses of the present invention, the PD-1 antagonist is a monoclonal antibody that specifically binds to human PD-1 and comprises (a) a heavy chain comprising SEQ ID NO: 12 and (b) a light chain comprising SEQ ID NO: 11.

In all of the above treatment methods, medicaments and uses, the PD-1 antagonist inhibits the binding of PD-L1 to PD-1, and in specific embodiments also inhibits the binding of PD-L2 to PD-1. In some embodiments of the above treatment methods, medicaments and uses, the PD-1 antagonist is a monoclonal antibody, or an antigen binding fragment thereof, that specifically binds to PD-1 or to PD-L1 and blocks the binding of PD-L1 to PD-1.

Table 2 below provides a list of the amino acid sequences of exemplary anti-PD-1 mAbs for use in the treatment method, medicaments and uses of the present invention.

TABLE 2
Exemplary PD-1 Antibody Sequences

| | | |
|---|---|---|
| Antibody | | SEQ |
| Feature | Amino Acid Sequence | ID NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 4 |
| Region | QQKPGQAPRLLIYLASYLESGVPARFSGSGSGTDFTLTIS | |
| | SLEPEDFAVYYCQHSRDLPLTFGGGTKVEIK | |
| Light Chain | EIVLTQSPATLSLSPGERATLSCRASKGVSTSGYSYLHWY | 5 |
| | QQKPGQAPRLLIYLASYLESGVPARFSGSGSGTDFTLTIS | |
| | SLEPEDFAVYYCQHSRDLPLTFGGGTKVEIKRTVAAPSVF | |
| | IFPPSDEQLKSGTASVVCLLNNFYPREAKVQWKVDNALQS | |
| | GNSQESVTEQDSKDSTYSLSSTLTLSKADYEKHKVYACEV | |
| | THQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWVR | 9 |
| Region | QAPGQGLEWMGGINPSNGGTNFNEKFKNRVTLTTDSSTT | |
| | TAYMELKSLQFDDTAVYYCARRDYRFDMGFDYWGQGTT | |
| | VTVSS | |
| Heavy | QVQLVQSGVEVKKPGASVKVSCKASGYTFTNYYMYWVR | 10 |
| Chain | QAPGQGLEWMGGINPSNGGTNFNEKFKNRVTLTTDSSTT | |
| | TAYMELKSLQFDDTAVYYCARRDYRFDMGFDYWGQGTTV | |
| | TVSSASTKGPSVFPLAPCSRSTSESTAALGCLVKDYFPEP | |
| | VTVSWNSGALTSGVHTFPAVLQSSGLYSLSSVVTVPSSSL | |
| | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWLN | |
| | GKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |

Nivolumab Light Chain

| | | |
|---|---|---|
| Light Chain | EIVLTQSPATLSLSPGERATLSCRASQSVSSYLAWYQQKPG | 11 |
| | QAPRLLIYDASNRATGIPARFSGSGSGTDFTLTISSLEPEDF | |
| | AVYYCQQSSNWPRTFGQGTKVEIKRTVAAPSVFIFPPSDE | |
| | QLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |

Nivolumab Heavy Chain

| | | |
|---|---|---|
| Heavy | QVQLVESGGGVVQPGRSLRLDCKASGITFSNSGMHWVRQ | 12 |
| Chain | APGKGLEWVAVIWYDGSKRYYADSVKGRFTISRDNSKNT | |
| | LFLQMNSLRAEDTAVYYCATNDDYWGQGTLVTVSSAST | |
| | KGPSVFPLAPCSRSTSESTAALGCLVKDYFPEPVTVSWNS | |
| | GALTSGVHTFPAVLQSSGLYSLSSVVTVPSSSLGTKTYTCN | |
| | VDHKPSNTKVDKRVESKYGPPCPPCPAPEFLGGPSVFLFPP | |
| | KPKDTLMISRTPEVTCVVVDVSQEDPEVQFNWYVDGVEV | |
| | HNAKTKPREEQFNSTYRVVSVLTVLHQDWLNGKEYKCK | |
| | VSNKGLPSSIEKTISKAKGQPREPQVYTLPPSQEEMTKNQV | |
| | SLTCLVKGFYPSDIAVEWESNGQPENNYKTTPPVLDSDGS | |
| | FFLYSRLTVDKSRWQEGNVFSCSVMHEALHNHYTQKSLS | |
| | LSLGK | |

In one embodiment, the anti-PD-1 antibody or antigen-binding fragment thereof comprises a heavy chain constant region, e.g. a human constant region, such as γ1, γ2, γ3, or γ4 human heavy chain constant region or a variant thereof. In another embodiment, the anti-PD-1 antibody or antigen-binding fragment thereof comprises a light chain constant region, e.g. a human light chain constant region, such as lambda or kappa human light chain region or a variant thereof. By way of example, and not limitation, the human heavy chain constant region can be γ4 and the human light chain constant region can be kappa. In an alternative embodiment, the Fc region of the antibody is γ4 with a Ser228Pro mutation (Schuurman, J et, al., Mol. Immunol. 38: 1-8, 2001). In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In another embodiment of the methods of treatment of the invention, the PD-1 antagonist is an antibody or antigen binding protein that has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the PD-1 antagonist is an antibody or antigen binding protein comprising variable light and variable heavy domains having up to 1, 2, 3, 4, or 5 or more amino acid substitutions, and exhibits specific binding to PD-1

### Methods of Treatment

In one aspect, the invention provides a method for treating a human patient with cancer which comprises:
(a) determining if a sample collected from a tumor from the patient has an altered SETD2 biomarker, and
(b) treating the patient with a treatment regimen that comprises a PD-1 antagonist if the altered SETD2 biomarker is present or treating the patient with a treatment regimen that does not include a PD-1 antagonist if the altered activity or amount is absent.

In one aspect, the invention provides a method for treating a human patient suffering from cancer who has been identified as having an altered SETD2 biomarker comprising treating the patient with a treatment regimen that comprises a PD-1 antagonist if the altered SETD2 biomarker is present or treating the patient with a treatment regimen that does not include a PD-1 antagonist if the altered activity or amount is absent.

In some embodiments, the altered SETD2 biomarker is an altered amount or altered level of a SETD2 biomarker. In some embodiments, the altered SETD2 biomarker is an altered activity of a SETD2 biomarker. In some embodiments, the altered SETD2 biomarker is an altered structure of a SETD2 biomarker.

In one embodiment, the altered activity or amount is reduced activity or amount of the SETD2 biomarker. In another embodiment, the altered activity is due to a SETD2 biomarker loss-of-function mutation. In one embodiment, the sample is a cancerous sample. In another embodiment, the altered activity is due to a SETD2 biomarker loss-of-function somatic mutation from a cancerous sample. In another embodiment, the altered activity or amount is reduced copy number of the SETD2 DNA. In another embodiment, the altered activity or amount is reduced level of the SETD2 mRNA. In another embodiment, the altered activity or amount is reduced amount of the SETD2 protein. In another embodiment, the altered activity or amount is reduced methylation level of the SETD2 protein substrate lysine-36 of histone H3.

In another aspect, the invention provides a method of treating a human patient with cancer which comprises:
(a) determining if a sample collected from the patient (e.g. a tumor sample) has reduced activity or amount of a SETD2 biomarker, and
(b) treating the patient with reduced activity or amount of the SETD2 biomarker a treatment regimen that comprises a therapeutically effective amount of a PD-1 antagonist.

In another aspect, the invention provides a method of treating a human patient with cancer which comprises:
(a) determining if a sample collected from the patient (e.g. a tumor sample) has an altered structure of a SETD2 biomarker, and
(b) treating the patient with an altered structure of the SETD2 biomarker a treatment regimen that comprises a therapeutically effective amount of a PD-1 antagonist.

In one embodiment, the sample is a cancerous sample. In another embodiment, the reduced activity or amount is reduced copy number of the SETD2 DNA, reduced amount of the SETD2 protein, reduced methylation level of the SETD2 protein substrate lysine-36 of histone H3, or reduced levels of SETD2 mRNA, as compared to a non-cancerous sample of the patient. In one embodiment, the copy number of the SETD2 DNA in the cancerous or germline sample is at least 50% reduced as compared to that in a control sample from a normal human individual. In one embodiment, the amount of the SETD2 protein in the cancerous sample is at least 20%, 30%, 40%, 50%, 60%, 70% or 80% reduced as compared to that in a non-cancerous sample in the patient. In another embodiment, the methylation level of the SETD2 protein substrate lysine-36 of histone H3 is at least 20%, 30%, 40%, 50%, 60%, 70% or 80% reduced as compared to that in a non-cancerous sample in the patient. In another embodiment, levels of the SETD2 mRNA is at least 20%, 30%, 40%, 50%, 60%, 70% or 80% reduced as compared to those in a non-cancerous sample in the patient. In one embodiment, the amount of the SETD2 protein is measured by an Immunohistochemistry (IHC) assay with an anti-SETD2 antibody. In another embodiment, the methylation level of its substrate lysine-36 of histone H3 is detected by an antibody to H3K36me3. In one embodiment, the amount of the SETD2 mRNA is measured by the nCounter®: Analysis System.

In another aspect, the invention provides a method of treating a human patient with cancer which comprises:
(a) determining if a sample collected from the patient has a somatic mutation in a SETD2 nucleic acid, and
(b) treating the patient with a treatment regimen that comprises a PD-1 antagonist if the somatic mutation is present.

In another aspect, the invention provides a method of treating a human patient with cancer which comprises:
(a) determining if a sample collected from the patient has a somatic mutation in a SETD2 nucleic acid, and
(b) treating the patient with a treatment regimen that comprises a PD-1 antagonist if the somatic mutation is present or treating the patient with a treatment regimen that does not include a PD-1 antagonist if the somatic mutation is absent.

In another aspect, the invention provides a method of identifying the likelihood of a cancer in a patient to be responsive to a PD-1 antagonist, the method comprising:
(a) obtaining or providing a sample from a patient having cancer, wherein the sample comprises nucleic acid molecules from the patient;
(b) identifying a somatic mutation in the SETD2 nucleic acid in the sample:
   wherein the somatic mutation in the SETD2 nucleic acid in the sample identifies the cancer as being more likely to be responsive to the PD-1 antagonist.

In one embodiment of the above methods, the somatic mutation is a loss-of-function mutation. In one embodiment of the above methods, the somatic mutation is a nonsynonymous mutation. In one embodiment of the above methods, the somatic mutation is a single-base substitution, multi-base substitutions, insertion mutation, deletion mutation, frameshift mutation, missense mutation, nonsense mutation, or splice-site mutation, or a combination thereof. In another embodiment, the somatic mutation is identified in a cancerous sample of the patient, and in specific embodiments the mutation is also not in a non-cancerous or germline sample of the patient. In one embodiment, nucleic acid (DNA) is extracted from cancerous samples. In specific embodiments, the Strelka method, Indelocator method or MuTect method can be used to detect the somatic mutations. In another embodiment, the mutation is from the SNP database and is also associated with cancer according to COSMIC (the Catalogue of Somatic Mutations in Cancer). See S. A. Forbes, G. Tang, N. Bindal, S. Bamford, E. Dawson, C. Cole, C. Y. Kok, M. Jia, R. Ewing, A. Menzies, J. W. Teague, M. R. Stratton, P. A. Futreal, COSMIC (the Catalogue of Somatic Mutations in Cancer); A resource to investigate acquired mutations in human cancer. Nucleic Acids Res. 38, D652-D657 (2010).

In one embodiment of the above methods, the patient who has reduced activity or amount of the SETD2 mutations comprises at least one of the mutations listed in Table 3. In a further embodiment, the patient who has reduced activity or amount of the SETD2 mutations, wherein the patient has at least one of the mutations listed in Table 3 is treated with a treatment regimen that comprises a therapeutically effective amount of a PD-1 antagonist.

In another aspect, the invention provides a method of identifying the likelihood of a cancer in a patient to be responsive to a PD-1 antagonist, the method comprising:
a) obtaining or providing a sample from a patient having cancer, wherein the sample comprises nucleic acid molecules from the patient;
b) determining the copy number of the SETD2 gene in the sample; and
c) comparing said copy number to that of a control sample,
   wherein a decreased copy number of the SETD2 gene in the sample and/or an increased copy number of the SETD2 gene having a loss of function mutation in the sample, relative to the control sample identifies the cancer as being more likely to be responsive to the a PD-1 antagonist.

In some embodiments, the control sample is a tissue sample from a patient that does not have cancer. In some embodiments, the control sample is a tissue sample from a patient that does have cancer, but is removed from a non-cancerous tissue or organ.

In a further aspect, the invention provides a method of identifying the likelihood of a cancer in a patient to be responsive to a PD-1 antagonist, the method comprising:
a) obtaining or providing a subject sample from a patient having cancer;
b) measuring the amount or activity of a SETD2 biomarker in the subject sample; and
c) comparing said amount or activity of the SETD2 biomarker to that in a control sample,
   wherein the absence of or a decreased amount or activity of the SETD2 biomarker in the subject sample, relative to the control sample, identifies the cancer as being more likely to be responsive to the PD-1 antagonist.

In one embodiment, step b) comprises measuring the amount of the SETD2 protein by an immunohistochemistry (IHC) assay. In another embodiment, step b) comprises measuring the activity of the SETD2 protein by detecting the methylation level of lysine-36 of its substrate histone H3.

In another embodiment, the subject sample is a cancerous sample from a patient, and the control sample is a non-cancerous sample from a patient.

Cancers that may be treated by the methods, medicaments and uses of the invention include, but are not limited to: Cardiac cancers including sarcoma (angiosarcoma, fibrosarcoma, rhabdomyosarcoma, liposarcoma), myxoma, rhabdomyoma, fibroma, lipoma and teratoma; lung cancers including bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal cancers including esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma) colorectal; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers including hepatoma (hepatocellular carcinoma), cholangiocarcinoma, hepatoblastoma, angiosarcoma, hepatocellular adenoma, hemangioma; bone cancers including osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers including blood (myeloid leukemia (acute and chronic), acute lymphoblastic leukemia, chronic lymphocytic leukemia, myeloproliferative diseases, multiple myeloma, myelodysplastic syndrome); hematopoietic tumors of the lymphoid lineage, including leukemia, acute lymphocytic leukemia, chronic lymphocytic leukemia, acute lymphoblastic leukemia, B-cell lymphoma, T-cell lymphoma, Hodgkin lymphoma, non-Hodgkin lymphoma, hairy cell lymphoma, mantle cell lymphoma, myeloma, and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma, and schwannomas; and other tumors, including melanoma, skin (non-melanoma) cancer, mesothelioma (cells), seminoma, teratocarcinoma, osteosarcoma, xeroderma pigmentosum, keratoacanthoma, thyroid follicular cancer and Kaposi's sarcoma. In one embodiment, the forgoing cancers are advanced, unresectable or metastatic.

In one embodiment, cancers that may be treated by the methods, medicaments and uses of the invention include, but are not limited to: melanoma, non-small cell lung cancer, head and neck cancer, urothelial cancer, breast cancer, gastric cancer, gastroesophageal junction adenocarcinoma, multiple myeloma, hepatocellular cancer, Merkel cell carcinoma, renal cell carcinoma, endometrial carcinoma, cutaneous squamous cell carcinoma, non-Hodgkin lymphoma, Hodgkin lymphoma, mesothelioma, ovarian cancer, small cell lung cancer, esophageal cancer, anal cancer, biliary tract cancer, colorectal cancer, cervical cancer, thyroid cancer, salivary cancer, prostate cancer, and glioblastoma. In one embodiment, the cancer is breast cancer which is triple negative breast cancer or ER+/HER2− breast cancer. In another embodiment, the cancer is non-Hodgkin lymphoma which is primary mediastinal B-cell lymphoma or diffuse large B-cell lymphoma.

In one embodiment, the cancer is renal cell carcinoma. In one embodiment, the renal cell carcinoma is advanced, unresectable or metastatic. In one embodiment, the cancer is clear cell renal cell carcinoma (ccRCC). In one embodiment, the cancer is non-clear cell renal cell carcinoma (nccRCC). In one embodiment, the cancer is bladder cancer. In one embodiment, the cancer is Stage IV. In another embodiment, the cancer is Stage III.

In one aspect of the foregoing embodiments, the patient with cancer progressed after anti-PD-1 or anti-PD-L1 treatment. In one embodiment, the patient with cancer has not received prior anti-PD-1 or anti-PD-L1 treatment. In one embodiment, the patient has received prior treatment for the cancer that does not include an anti-PD-1 or anti-PD-L1 treatment.

The methods, medicaments and uses of the invention may also comprise one or more additional therapeutic agents. The additional therapeutic agent may be, e.g., a chemotherapeutic, a biotherapeutic agent (including but not limited to antibodies to VEGF. EGFR. Her2/neu. VEGF receptors, other growth factor receptors. CD20, CD40, CD-40L, GITR. CTLA-4. OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

Each therapeutic agent in the methods, medicaments and uses of the invention may be administered either alone or in a medicament (also referred to herein as a pharmaceutical composition) that comprises the therapeutic agent and one or more pharmaceutically acceptable carriers, excipients and diluents, according to standard pharmaceutical practice.

Each therapeutic agent in the methods, medicaments and uses of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew. Chem. Intl. Ed. Engl., 33:183-186 (1994); dynemicin, including dynemicin A; bisphosphonates, such as clodronate: an esperamicin; as well as neocarzinostatin chromophore and related chromoprotein enedivne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin. 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan: vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum: etoposide (VP-16); ifosfamide; mitoxantrone; vincristine; vinorelbine; novantrone; teniposide; edatrexate; daunomycin; aminopterin: xeloda: ibandronate; CPT-11: topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

In some embodiments, at least one of the therapeutic agents in the methods, medicaments and uses of the invention is administered using the same dosage regimen (dose, frequency and duration of treatment) that is typically employed when the agent is used as monotherapy for treating the same cancer. In other embodiments, the patient receives a lower total amount of at least one of the therapeutic agents in the methods, medicaments and uses than when the agent is used as monotherapy, e.g., smaller doses, less frequent doses, and/or shorter treatment duration.

Each small molecule therapeutic agent in the methods, medicaments and uses of the invention can be administered orally or parenterally, including the intravenous, intramuscular, intraperitoneal, subcutaneous, rectal, topical, and transdermal routes of administration.

The methods, medicaments and uses of the invention may be used prior to or following surgery to remove a tumor and may be used prior to, during or after radiation therapy.

In some embodiments, the method of the invention is administered to a patient who has not been previously treated with a biotherapeutic or chemotherapeutic agent, i.e., is treatment-naïve. In other embodiments, the therapy is administered to a patient who failed to achieve a sustained response after prior therapy with a biotherapeutic or chemotherapeutic agent, i.e., is treatment-experienced.

In some embodiments, a therapy of the invention is used to treat a tumor that is large enough to be found by palpation or by imaging techniques well known in the art, such as MRI, ultrasound, or CAT scan.

Selecting a dosage regimen (also referred to herein as an administration regimen) for a therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in a combination depends in part on the particular therapeutic agent, the severity of the cancer being treated, and patient characteristics. Guidance in selecting appropriate doses of antibodies, cytokines, and small molecules are available. See. e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY: Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792: Beniaminovitz et al. (2000) New Engl. J. Med. 342:613-619: Ghosh et al. (2003) New Engl. J. Med. 348:24-32: Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the therapy.

In some embodiments that employ an anti-human PD-1 mAb as the PD-1 antagonist in the methods, medicaments and uses of the invention, the dosing regimen comprises administering the anti-human PD-1 mAb at a dose of 1, 2, 3, 5 or 10 mg/kg at intervals of about 14 days (±2 days) or about 21 days (±2 days) or about 30 days (±2 days) throughout the course of treatment. In other embodiments, the dosage is not based on the weight of the patient, e.g. flat dosages of 200 mg, 240 mg. 300 mg, 360 mg, 400 mg, 480 mg.

In other embodiments that employ an anti-human PD-1 mAb as the PD-1 antagonist in the methods, medicaments and uses of the invention, the dosing regimen comprises administering the anti-human PD-1 mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject is administered an intravenous (IV) infusion or subcutaneous injection of a medicament comprising any of the PD-1 antagonists described herein.

In one embodiment of the invention, the PD-1 antagonist in the therapy is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg/kg Q3W. In some embodiments, nivolumab is administered at a dose selected from the group consisting of 240 mg Q2W, 360 mg Q3W and 480 mg Q4W.

In another embodiment of the invention, the PD-1 antagonist in the therapy is pembrolizumab, or a pembrolizumab variant, that is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W and flat-dose equivalents of any of these doses, i.e., such as 200 mg Q3W or 400 mg Q6W. In some embodiments, pembrolizumab is provided as a liquid medicament that comprises 25 mg/mL pembrolizumab, 7% (w/v) sucrose, 0.02% (w/v) polysorbate 80 in 10 mM histidine buffer pH 5.5. In other embodiments, pembrolizumab is provided as a liquid medicament that comprises about 125 to about 200 mg/mL of pembrolizumab, or an antigen binding fragment thereof; about 10 mM histidine buffer; about 10 mM L-methionine, or a pharmaceutically acceptable salt thereof; about 7% (w/v) sucrose; and about 0.02% (w/v) polysorbate 80.

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further embodiments, the method comprises administering 2 mg/kg of pembrolizumab to the patient about every three weeks. In particular embodiments, the patient is a pediatric patient.

In some embodiments, the selected dose of pembrolizumab is administered by IV infusion. In one embodiment, the selected dose of pembrolizumab is administered by IV infusion over a time period of between 25 and 40 minutes, or about 30 minutes. In other embodiments, the selected dose of pembrolizumab is administered by subcutaneous injection.

In some embodiments, the patient is treated with the therapy for at least 24 weeks, e.g., eight 3-week cycles. In some embodiments, treatment with the therapy continues until the patient exhibits evidence of PD or a CR.

In one aspect of the invention, the PD-1 antagonist in included in a pharmaceutical composition with a pharmaceutically acceptable carrier or diluent and may include additional pharmaceutically acceptable excipients.

Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbitol, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in some embodiments, the pharmaceutical composition is prepared under sterile conditions and is in a single use container, and thus does not necessitate inclusion of a preservative.

In some embodiments, a medicament comprising an anti-PD-1 antibody as the PD-1 antagonist may be provided as a liquid formulation or prepared by reconstituting a lyophilized powder with sterile water for injection prior to use. PCT International application publ, no. WO 2012/135408 describes the preparation of liquid and lyophilized medicaments comprising pembrolizumab that are suitable for use in the present invention. In some embodiments, a medicament comprising pembrolizumab is provided in a glass vial that contains about 100 mg of pembrolizumab in 4 ml of solution. Each 1 mL of solution contains 25 mg of pembrolizumab and is formulated in: L-histidine (1.55 mg), polysorbate 80 (0.2 mg), sucrose (70 mg), and Water for Injection, USP. The solution requires dilution for IV infusion.

In one aspect, the invention comprises a kit for assaying tumor samples to determine if a human patient with cancer has reduced activity or amount of the SETD2 biomarker.

In one embodiment, the utility of the claimed drug products and treatment methods does not require that the claimed or desired effect is produced in every cancer patient; all that is required is that a clinical practitioner, when applying his or her professional judgment consistent with all applicable norms, decides that there is reasonable chance of achieving the claimed effect of treating a given patient according to the claimed method or with the claimed composition or drug product.

### Biomarker Measurement

In some embodiments, biomarker amount and/or activity measurement(s) in a sample from a subject is compared to a predetermined control (standard) sample. The sample from the subject is typically from a diseased tissue, such as cancer cells or tissues. The control sample can be from the same subject or from a different subject. The control sample is typically a normal, non-diseased sample. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment, evaluate a response to a PD-1 antagonist therapy, and/or evaluate a response to a combination therapy with a PD-1 antagonist. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., biomarker copy numbers, level, and/or activity).

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement s) can be obtained from a previous assessment of the same patient. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention, the change of biomarker amount and/or activity measurement s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive.

Biological samples can be collected from a variety of sources from a patient including a body fluid sample, cell sample, or a tissue (tumor) sample comprising nucleic acids and/or proteins. In one embodiment, the subject and/or control sample is selected from the group consisting of cells, paraffin embedded tissues, biopsies, whole blood, serum, and plasma. In one embodiment, the sample is serum, plasma, or urine. In another embodiment, the sample is a cancerous sample.

Each of the steps of obtaining a tissue sample, preparing one or more tissue sections therefrom for assaying gene expression, performing the assay, and analyzing the results may be performed by separate individuals at separate locations. For example, a surgeon may obtain by biopsy a tissue sample from a cancer patient's tumor and then send the tissue sample to a pathology lab, and a technician in the lab may fix the tissue sample and then prepare one or more slides, each with a single tissue section, for the assay. The slide(s) may be assayed soon after preparation, or stored for future assay. The lab that prepared a tissue section may conduct the assay or send the slide(s) to a different lab to conduct the assay.

### Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

Preferably, the accuracy of the result provided by a diagnostic method of the invention is one that a skilled artisan or regulatory authority would consider suitable for the particular application in which the method is used.

### A. Methods for Detection of Biomarker Gene Mutation

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation). The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. Alternative alleles can be identified by sequencing the gene of interest in a number of different individuals. This can be readily carried out by using hybridization probes to identify the same genetic locus in a variety of individuals.

Somatic nucleotide substitutions in cancer are often difficult to identify. First, they occur at a very low frequency in the genome, ranging from 0.1 to 100 mutations per megabase, depending on tumor type. Second, the alterations may be present only in a small fraction of the DNA molecules originating from the specific genomic locus for reasons including: contaminating normal cells in the analyzed sample; local copy-number variation within the cancer genome; and presence of a mutation within only a sub-population of the tumor cells. The sensitivity and specificity of any somatic-mutation calling method varies along the genome. They depend on several factors, including the following: depth of sequence coverage in the tumor and a patient-matched normal sample; the local sequencing error rate; the allelic fraction of the mutation; and the evidence thresholds used to declare a mutation. In one embodiment, the MuTect somatic point mutation caller can be used, which provides high sensitivity for low allelic fractions and specificity (K. Cibulskis, et al. Nat. Biotechnol. 31, 213-219 (2013)).

In other embodiments, the Strelka method can be used to identify somatic insertions and deletions across the whole exome by alignment of tumor and normal sequences (Saunders et al. (2012) Bioinformatics 28:1811-1817). In other embodiments, the Indelocator method can be used to identify small somatic insertions and deletions across the whole exome by alignment of tumor and normal sequences (Cancer Genome Atlas Research (2011) Nature 474:609-615). In specific embodiments, the combination of the Strelka method and Indelocator method can be used.

### B. Methods for Detection of Copy Number

Methods of evaluating the copy number of a biomarker nucleic acid are well known to those of skill in the art. The presence or absence of chromosomal gain or loss can be evaluated simply by a determination of copy number of the regions or markers identified herein. In one embodiment, a biological sample is tested for the presence of copy number changes in genomic loci containing the genomic marker.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well known in the art to detect RNA can be used, such that higher or lower expression relative to an appropriate control (e.g., a non-amplified portion of the same or related cell tissue, organ, etc.) provides an estimate of the relative copy number of the target nucleic acid. An alternative means for determining genomic copy number is in situ hybridization (e.g., Angerer (1987) et al. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary.

Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise CDNAs, genomic DNAs, oligonucleotides (e.g., to detect single nucleotide polymorphisms) and the like. Array-based CGH may also be performed with single-color labeling (as opposed to labeling the control and the possible cancerous sample with two different dyes and mixing them prior to hybridization, which will yield a ratio due to competitive hybridization of probes on the arrays). In single color CGH, the control is labeled and hybridized to one array and absolute signals are read, and the possible cancerous sample is labeled and hybridized to a second array (with identical content) and absolute signals are read. Copy number difference is calculated based on absolute signals from the two arrays. Methods of preparing immobilized chromosomes or arrays and performing comparative genomic hybridization are well known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci, USA 85: 9138-9142; EPO Pub. No. 430,402; Methods in Molecular Biology, Vol. 33; In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl. Acad. Sci, USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green. Other suitable amplification methods include, but are not limited to, ligase chain reaction (LCR) (see Wu and Wallace (1989) Genomics 4: 560, Landegren, et al. (1988) Science 241: 1077, and Barringer et al. (1990) Gene 89: 117), transcription amplification (Kwoh, et al. (1989) Proc. Natl. Acad. Sci, USA 86: 1173), self-sustained sequence replication (Guatelli, et al. (1990) Proc. Nat. Acad. Sci, USA 87: 1874), dot PCR, and linker adapter PCR, etc. Loss of heterozygosity (LOH) and major copy proportion (MCP) mapping (Wang, Z. C., et al. (2004) Cancer Res 64(1); 64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

### C. Methods for Detection of Biomarker Protein Expression

The activity or level of a biomarker protein can be detected and/or quantified by detecting or quantifying the expressed polypeptide. The polypeptide can be detected and quantified by any of a number of means well known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to the PD-1 antagonist therapy. Any method known in the art for detecting polypeptides can be used. Such methods include, but are not limited to, immunodiffusion, immunoelectrophoresis, radioimmunoassay (RIA), enzyme-linked immunosorbent assay's (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn, pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Other techniques may be used to detect biomarker protein according to a practitioner's preference based upon the present disclosure. One such technique is Western blotting (Towbin et at., Proc. Nat. Acad. Sci, USA 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy. 30) Alteration of SETD2 protein expression may be detected with an anti-SETD2 antibody (for example, Abcam, ab113642) in an IHC assay of a tumor tissue section or by flow cytometry. Alternatively, SETD2 protein expression by tumor cells may be detected by PET imaging, using a binding agent (e.g., antibody fragment, affibody and the like) that specifically binds to the SETD2 protein.

In some embodiments, a level of SETD2 expression (protein and/or mRNA) by malignant cells within a tumor is determined to be “reduced” based on comparison with the level of SETD2 expression (protein and/or mRNA) by an appropriate control. For example, a control SETD2 protein or mRNA expression level may be the level quantified in non-cancerous cells of the same tissue as the tumor tissue.

### D. Assaying Cancerous Samples for Gene Expression of Biomarkers

A gene expression level is determined in a sample of tumor tissue removed from a subject. The tumor may be primary or recurrent, and may be of any type (as described above), any stage (e.g., Stage I, II, III, or IV or an equivalent of other staging system), and/or histology.

The cancerous sample can be obtained by a variety of procedures including, but not limited to, surgical excision, aspiration or biopsy. The tissue sample may be sectioned and assayed as a fresh specimen: alternatively, the tissue sample may be frozen for further sectioning. In some embodiments, the tissue sample is preserved by fixing and embedding in paraffin or the like.

The tumor tissue sample may be fixed by conventional methodology, with the length of fixation depending on the size of the tissue sample and the fixative used. Neutral buffered formalin, glutaraldehyde, Bouin's and paraformaldehyde are nonlimiting examples of fixatives. In some embodiments, the tissue sample is fixed with formalin. In some embodiments, the fixed tissue sample is also embedded in paraffin to prepare an FFPE tissue sample.

Typically, the tissue sample is fixed and dehydrated through an ascending series of alcohols, infiltrated and embedded with paraffin or other sectioning media so that the tissue sample may be sectioned. Alternatively, the tumor tissue sample is first sectioned and then the individual sections are fixed.

In some embodiments, the gene expression level of SETD2 for a tumor is determined using FFPE tissue sections of about 3-4 millimeters, and preferably 4 micrometers, which are mounted and dried on a microscope slide.

Once a suitable sample of tumor tissue has been obtained, it is analyzed to quantitate the RNA expression level for SETD2. The phrase “determine the RNA expression level of a gene” as used herein refers to detecting and quantifying RNA transcribed from that gene. The term “RNA transcript” includes mRNA transcribed from the gene, and/or specific spliced variants thereof and/or fragments of such mRNA and spliced variants.

A person skilled in the art will appreciate that a number of methods can be used to isolate RNA from the tissue sample for analysis. For example, RNA may be isolated from frozen tissue samples by homogenization in guanidinium isothiocyanate and acid phenol-chloroform extraction. Commercial kits are available for isolating RNA from FFPE samples. If the cancerous sample is an FFPE tissue section on a glass slide, it is possible to perform gene expression analysis on whole cell lysates rather than on isolated total RNA.

Persons skilled in the art are also aware of several methods useful for detecting and quantifying the level of RNA transcripts within the isolated RNA or whole cell lysates. Quantitative detection methods include, but are not limited to, arrays (i.e., microarrays), quantitative real time PCR (RT-PCR), multiplex assays, nuclease protection assays, and Northern blot analyses. Generally, such methods employ labeled probes that are complimentary to a portion of each transcript to be detected. Probes for use in these methods can be readily designed based on the known sequences of the genes and the transcripts expressed thereby. In some embodiments, a probe for detecting a transcript of SETD2 is designed to specifically hybridize to a target region for that gene. Suitable labels for the probes are well-known and include, e.g., fluorescent, chemiluminescent and radioactive labels.

In some embodiments, assaying a cancerous sample for expression of SETD2 employs detection and quantification of RNA levels in real-time using nucleic acid sequence based amplification (NASBA) combined with molecular beacon detection molecules. NASBA is described, e.g., in Compton J., Nature 350 (6313); 91-92 (1991). NASBA is a single-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in the first step, such that the reaction is cyclic.

In other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In yet other embodiments, the assay format employs direct mRNA capture with branched DNA (QuantiGene™, Panomics) or Hybrid Capture™ (Digene).

One example of an array technology suitable for use in measuring expression of the genes in gene expression platform of the invention is the Array Plate™ assay technology sold by HTG Molecular, Tucson Arizona, and described in Martel, R. R., et al., Assay and Drug Development Technologies 1(1); 61-71, 2002. In brief, this technology combines a nuclease protection assay with array detection. Cells in microplate wells are subjected to a nuclease protection assay. Cells are lysed in the presence of probes that bind targeted mRNA species. Upon addition of SI nuclease, excess probes and unhybridized mRNA are degraded, so that only mRNA:probe duplexes remain. Alkaline hydrolysis destroys the mRNA component of the duplexes, leaving probes intact. After the addition of a neutralization solution, the contents of the processed cell culture plate are transferred to another Array Plate™ called a programmed Array Plate™, Array Plates™ contain a 16-element array at the bottom of each well. Each array element comprises a position-specific anchor oligonucleotide that remains the same from one assay to the next. The binding specificity of each of the 16 anchors is modified with an oligonucleotide, called a programming linker oligonucleotide, which is complementary at one end to an anchor and at the other end to a nuclease protection probe. During a hybridization reaction, probes transferred from the culture plate are captured by immobilized programming linker. Captured probes are labeled by hybridization with a detection linker oligonucleotide, which is in turn labeled with a detection conjugate that incorporates peroxidase. The enzyme is supplied with a chemiluminescent substrate, and the enzyme-produced light is captured in a digital image. Light intensity at an array element is a measure of the amount of corresponding target mRNA present in the original cells.

Techniques for detecting and measuring mRNA expression include RT-PCR, real-time quantitative RT-PCR. RNAseq, and the Nanostring platform (J. Clin. Invest. 2017:127(8); 2930-2940). The level of mRNA expression may be compared to the mRNA expression levels of a reference sample in quantitative RT-PCR.

By way of further example, DNA microarrays can be used to measure gene expression. In brief, a DNA microarray, also referred to as a DNA chip, is a microscopic array of DNA fragments, such as synthetic oligonucleotides, disposed in a defined pattern on a solid support, wherein they are amenable to analysis by standard hybridization methods (see Schena, BioEssays 18:427 (1996)). Exemplary microarrays and methods for their manufacture and use are set forth in T. R. Hughes et al., Nature Biotechnology 9:342-347 (2001). A number of different microarray configurations and methods for their production are known to those of skill in the art and are disclosed in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 6,022,963; 6,077,674; and U.S. Pat. No. 6,156,501; Shena, et al., Tibtech 6:301-306, 1998; Duggan, et al., Nat. Genet. 2:10-14, 1999; Bowtell, et al., Nat. Genet. 21:25-32, 1999; Lipshutz, et al., Nat. Genet. 21:20-24, 1999; Blanchard, et al., Biosensors and Bioelectronics 77:687-90, 1996; Maskos, et al., Nucleic Acids Res. 2:4663-69, 1993; and Hughes, et al., Nat. Biotechnol. 79:342-347, 2001. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; the disclosures of which are herein incorporated by reference.

In one embodiment, an array of oligonucleotides may be synthesized on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as “DNA chips” or very large scale immobilized polymer arrays (“VLSIPSR” arrays), may include millions of defined probe regions on a substrate having an area of about 1 cm² to several cm², thereby incorporating from a few to millions of probes (see, e.g., U.S. Pat. No. 5,631,734).

To compare expression levels, labeled nucleic acids may be contacted with the array under conditions sufficient for binding between the target nucleic acid and the probe on the array. In one embodiment, the hybridization conditions may be selected to provide for the desired level of hybridization specificity; that is, conditions sufficient for hybridization to occur between the labeled nucleic acids and probes on the microarray.

Hybridization may be carried out in conditions permitting essentially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al. (Laboratory Techniques in Biochemistry and Molecular Biology, Vol. 24; Hybridization With Nucleic Acid Probes, P. Tijssen, ed.; Elsevier, N.Y. (1993)). The methods described above will result in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent Gene Array® Scanner. This scanner is controlled from a system computer with an interface and easy-to-use software tools. The output may be directly imported into or directly read by a variety of software applications. Exemplary scanning devices are described in, for example, U.S. Pat. Nos. 5,143,854 and 5,424,186.

An assay method useful for measuring transcript abundance of SETD2 utilizes the nCounter® Analysis System marketed by NanoString® Technologies (Seattle, Washington USA). This system, which is described by Geiss et al., Nature Biotechnol. 2(3); 317-325 (2008), utilizes a pair of probes, namely, a capture probe and a reporter probe, each comprising a 35- to 50-base sequence complementary to the transcript to be detected. The capture probe additionally includes a short common sequence coupled to an immobilization tag, e.g. an affinity tag that allows the complex to be immobilized for data collection. The reporter probe additionally includes a detectable signal or label, e.g. is coupled to a color-coded tag. Following hybridization, excess probes are removed from the sample, and hybridized probe/target complexes are aligned and immobilized via the affinity or other tag in a cartridge. The samples are then analyzed, for example using a digital analyzer or other processor adapted for this purpose. Generally, the color-coded tag on each transcript is counted and tabulated for each target transcript to yield the expression level of each transcript in the sample. This system allows measuring the expression of hundreds of unique gene transcripts in a single multiplex assay using capture and reporter probes designed by NanoString.

In measuring expression of the SETD2, the absolute expression in a cancerous sample is compared to a control: for example, the control can be the average level of expression of SETD2, in a pool of subjects.

### General Methods

Standard methods in molecular biology are described Sambrook, Fritsch and Maniatis (1982 & 1989 2^{nd} Edition, 2001 3^{rd} Edition) Molecular Cloning. A Laboratory Manual, Cold Spring Harbor Laboratory Press, Cold Spring Harbor, NY; Sambrook and Russell (2001) Molecular Cloning. 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology. Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science. Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science. Vol. 2, John Wiley and Sons, Inc., New York: Ausubel, et al. (2001) Current Protocols in Molecular Biology. Vol. 3, John Wiley and Sons, Inc., NY, NY, pp. 16.0.5-16.22.17; Sigma-Aldrich, Co. (2001) Products for Life Science Research, St. Louis, MO: pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology. Vol. 1, John Wiley and Sons, Inc., New York: Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology. Vol. 4, John Wiley, Inc., New York).

Monoclonal, polyclonal, and humanized antibodies can be prepared (see, e.g., Shepherd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160: 1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York: Kay et al. (1996) Phage Display of Peptides and Proteins: A Laboratory Manual, Academic Press, San Diego, CA: de Bruin et al. (1999) Nature Biotechnol. 17:397-399). Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry. 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR: Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY). Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Examples

### Example 1: Pembrolizumab Therapy in Renal Cell Carcinoma Patients

A Phase II, open-label, multicenter, global trial to evaluate the efficacy and safety of pembrolizumab as a first-line treatment for advanced/metastatic renal cell carcinoma (mRCC) was conducted. The safety and efficacy of pembrolizumab (200 mg every 3 weeks [Q3W]) was studied in advanced RCC (clear cell (cc) RCC [Cohort A] and non-clear cell (ncc) RCC [Cohort B]). The primary objective of the study for both cohorts was to evaluate the objective response rate (ORR), using Response Evaluation Criteria in Solid Tumors version 1.1 (RECIST 1.1).

### Example 2: Whole Exome Sequencing

Whole-exome sequence (WES) was performed on formalin-fixed, paraffin-embedded pretreatment tumor samples using either ImmunoSELECT™-RUO or ACE Cancer Exome™. After pathology assessment, the tissue was either macro-dissected with a fresh scalpel from the marked tumor area (tissue containing <20% tumor) or scraped from the entire section and transferred to a 1.5-mL tube containing 200 μL of 100% ethanol. DNA was isolated using the QIAamp DNA FFPE Tissue Kit (Qiagen). Tumor DNA was quantitated using the Qubit assay (Invitrogen); quality was assessed using the QuantideX qPCR DNA QC Assay (Assuragen). WES was performed on matched normal DNA from whole blood collected in a PAXgene DNA Tube (Qiagen) at clinical sites and stored at −20° ° C., or −70/80° ° C., until processed in an approved central laboratory identified by the sponsor. The Chemagic STAR DNA Blood Kit (Perkin Elmer) run on either a Hamilton Chemagic STAR or Perkin Elmer Chemagic 360 automated instrument was used to extract DNA in a final volume of 500 μL or 1.0 mL. Extracted DNA was subjected to volume and concentration determination and ultraviolet and visible spectral analysis to assess purity. Whole-exome sequence reads were aligned to reference human genome GRCh37 by using Burrows-Wheeler Aligner (BWA-MEM) (H. Li et al., Fast and accurate short read alignment with Burrows-Wheeler transform. Bioinformatics 25, 1754-1760 (2009). doi: 10.1093/bioinformatics/btp324) followed by preprocessing steps including duplicate marking, indel realignment, and base recalibration with Picard (v1.114) and GATK (Genome Analysis Toolkit, v4) (McKenna, Aaron et al. “The Genome Analysis Toolkit: a MapReduce framework for analyzing next-generation DNA sequencing data.” Genome research vol. 20, 9 (2010); 1297-303, doi: 10.1101/gr. 107524.110) to generate analysis-ready Binary Alignment Map (BAM) files, which stores the aligned sequencing data in a compressed binary representation. MuTect2 was used to generate somatic single nucleotide variation (SNV) or Indel (Insertion or Deletions up to 50 base pairs) calls using default parameters by comparing BAM files from tumor and matched normal sample of the patient (Cibulskis, Kristian et al. “Sensitive detection of somatic point mutations in impure and heterogeneous cancer samples.” Nature Biotechnology vol. 31, 3 (2013); 213-9, doi: 10.1038/nbt.2514; Cancer Genome Atlas Research (2011) Nature 474:609-615). If a mutation is found in both tumor and normal samples, then it could be a germline mutation or sequencing error, and therefore these mutations are removed from the mutations listed in Table 3. Mutations present in the Single Nucleotide Polymorphism Database (dbSNP, v141, www.ncbi.nlm.nih.gov/books/NBK21088/) (Sherry, S T et al. “dbSNP-database for single nucleotide polymorphisms and other classes of minor genetic variation.” Genome research vol. 9, 8 (1999); 677-9) but not in the Catalogue of Somatic Mutations in Cancer (COSMIC, v68) (Forbes, Simon A et al., COSMIC (the Catalogue of Somatic Mutations in Cancer); A resource to investigate acquired mutations in human cancer. Nucleic Acids Res. 38, D652-D657 (2010)), doi: 10.1093/nar/gkp995) were filtered out. The Single Nucleotide Polymorphism database (dbSNP) is a public-domain archive for a broad collection of simple genetic polymorphisms. This collection of polymorphisms includes single-base nucleotide substitutions (SNPs), small-scale multi-base deletions or insertions (also called deletion insertion polymorphisms or DIPs), and retroposable element insertions and microsatellite repeat variations (also called short tandem repeats or STRs). Mutations with mutant reads of <4 in tumor samples were also eliminated due to possibility of false positives. Mutations were annotated using Ensembl Variant Effect Predictor (McLaren, William et al. “The Ensembl Variant Effect Predictor.” Genome biology vol. 17,1 122. 6 Jun. 2016, doi: 10.1186/s13059-016-0974-4), and SETD2 biomarker mutations are listed in Table 3.

Copy ratios were calculated for each captured target by dividing the tumor coverage by the median coverage obtained in a set of reference normal samples. The resulting copy ratios were segmented using the circular binary segmentation algorithm (Olshen et al. (2004) Biostatistics 5:557-572). Allelic copy number alterations were called while taking into account sample-specific overall chromosomal aberrations (focality) (Brastianos et al. (2015) Cancer Discov. 5: 1164-1177). Inference of mutational clonality, tumor purity, and tumor ploidy was accomplished with ABSOLUTE (Carter et al. (2012) Nat Biotechnol. 30:413-421). Samples having estimated tumor purity greater than 10% were included in the final analysis.

TABLE 3
SETD2 biomarker mutations

| | | | | | | CDNA_ | Protein_ | | |
|---|---|---|---|---|---|---|---|---|---|
| | | | | | Conse- | posi- | posi- | Amino_ | |
| cohort | CHROM | POS | >REF | QALT | quence | tion | tion | acids | Codons |
| A | 3 | 47125868 | A | | missense | 5445 | 1801 | I/R | aTa/ |
| | | | | | _variant | | | | aGa |
| A | 3 | 47161824 | TC | T | frameshift | 4344 | 1434 | G/X | gGa/ |
| | | | | | _variant | | | | ga |
| A | 3 | 47165090 | C | A | Nonsense | 1079 | 346 | E/* | Gaa/ |
| | | | | | _variant | | | | Taa |
| A | 3 | 47098867 | TTC | T | frameshift | 6448- | 2135- | QK/Q | caGAaa/ |
| | | | | | _variant | 6449 | 2136 | X | caaa |
| A | 3 | 47127707 | CT | C | frameshift | 5417 | 1792 | S/X | Agt/gt |
| | | | | | _variant | | | | |
| A | 3 | 47155459 | T | C | missense | 4665 | 1541 | N/S | aAt/ |
| | | | | | _variant | | | | aGt |
| A | 3 | 47158117 | CAATCATGA | C | inframe_ | 4616- | 1525- | LMI/— | CTCA |
| | | | G | | deletion | 4624 | 1527 | | TGATT/— |
| A | 3 | 47143009 | T | TA | frameshift | 4996- | 1651- | —/X | —/T |
| | | | | | _variant | 4997 | 1652 | | |
| A | 3 | 47164786 | GA | G | frameshift | 1382 | 447 | S/X | Tct/ct |
| | | | | | _variant | | | | |
| A | 3 | 47125873 | C | G | splice_ | | | | |
| | | | | | acceptor | | | | |
| | | | | | _variant | | | | |
| A | 3 | 47129710 | C | A | nonsense | 5213 | 1724 | E/* | Gaa/ |
| | | | | | variant | | | | Taa |
| A | 3 | 47155393 | CCCCAGCC | C | frameshift | 4706- | 1555- | ILTE | ATACTCAC |
| | | | TTTCTTTTC | | _variant | 4730 | 1563 | KK | AGAAAA |
| | | | TGTGAGTAT | | | | | GWG/ | GAAAGGC |
| | | | | | | | | X | TGGGgc/gc |
| A | 3 | 47165219 | TA | T | frameshift | 949 | 302 | C/X | tgT/tg |
| | | | | | _variant | | | | |
| A | 3 | 47059149 | G | GT | frameshift | 7554- | 2504 | D/EX | gac/gaAc |
| | | | | | _variant | 7555 | | | |
| A | 3 | 47164171 | A | T | nonsense | 1998 | 652 | L/* | tTa/tAa |
| | | | | | variant | | | | |
| A | 3 | 47059165 | AT | A | frameshift | 7538 | 2499 | I/X | Att/tt |
| | | | | | _variant | | | | |
| A | 3 | 47161689 | A | AT | frameshift | 4479- | 1479 | N/KX | aat/aaAt |
| | | | | | _variant | 4480 | | | |
| A | 3 | 47103652 | C | T | splice_ | | | | |
| | | | | | donor | | | | |
| | | | | | _variant | | | | |
| A | 3 | 47125259 | TC | T | frameshift | 6053 | 2004 | D/X | Gat/at |
| | | | | | _variant | | | | |
| B | 3 | 47088046 | TC | T | frameshift | 7071 | 2343 | G/X | gGa/ga |
| | | | | | _variant | | | | |
| B | 3 | 47108602 | A | C | missense | 6110 | 2023 | Y/I | Tat/Gat |
| | | | | | _variant | | | | |
| B | 3 | 47164137 | TA | T | frameshift | 2031 | 663 | L/X | tTa/ta |
| | | | | | _variant | | | | |
| B | 3 | 47098795 | GGGGCATTA | G | frameshift | 6497- | 2152- | YDSL | TATGACTCT |
| | | | TAACCAAGAG | | _variant | 6521 | 2160 | G | CTTGGT |
| | | | AGTCATA | | | | | YNAP | TATAATGC |
| | | | | | | | | /X | CCcg/cg |
| B | 3 | 47164686 | G | T | nonsense | 1483 | 480 | Y/* | taC/taA |
| | | | | | variant | | | | |
| B | 3 | 47098478 | G | A | nonsense | 6839 | 2266 | Q/* | Cag/Tag |
| | | | | | variant | | | | |
| B | 3 | 47129670 | C | CA | frameshift | 5252- | 1737 | S/MX | agc/aTgc |
| | | | | | _variant | 5253 | | | |
| B | 3 | 47129671 | T | G | missense | 5252 | 1737 | S/R | Agc/Cgc |
| | | | | | _variant | | | | |
| B | 3 | 47161845 | AACTTTC | A | frameshift | 4295- | 1418- | DGELQ | GATGGTG |
| | | | TTTCT | | _variant | 4323 | 1427 | DR | AGCTTCA |
| | | | GTCCT | | | | | KKV/X | GGACAGAA |
| | | | AGCTCA | | | | | | AGAAAGTt/t |
| | | | CCATC | | | | | | |
| B | 3 | 47163373 | G | GA | frameshift | 2795- | 918 | S/FX | tca/ |
| | | | | | _variant | 2796 | | | tTca |
| B | 3 | 47098318 | A | AT | frameshift | 6998- | 2319 | I/NX | att/ |
| | | | | | _variant | 6999 | | | aAtt |
| B | 3 | 47139458 | C | T | missense | 5172 | 1710 | R/H | cGt/ |
| | | | | | _variant | | | | cAt |
| B | 3 | 47165282 | C | CT | frameshift | 886- | 281- | —/X | —/A |
| | | | | | _variant | 887 | 282 | | |
| B | 3 | 47061267 | CT | C | frameshift | 7456 | 2471 | K/X | aaA/aa |
| | | | | | _variant | | | | |
| B | 3 | 47162755 | GCT | G | frameshift | 3412- | 1123- | KA/N | aaAGcc/ |
| | | | | | _variant | 3413 | 1124 | X | aacc |
| B | 3 | 47061252 | CT | C | frameshift | 7471 | 2476 | K/X | aaA/aa |
| | | | | | _variant | | | | |
| B | 3 | 47143046 | C | T | splice_ | | | | |
| | | | | | acceptor | | | | |
| | | | | | _variant | | | | |

TABLE 4
Abbreviations in Table 3

| | Description |
|---|---|
| CHROM | chromosome of the mutation |
| POS | Genomic positon of the base pair |
| REF | reference allele |
| ALT | mutant allele |
| Consequence | consequence type of this variant |
| cDNA_position | relative position of base pair in cDNA sequence |
| Protein_position | relative position of amino acid in protein |
| Amino_acids | only given if the variant affects the protein-coding sequence; |
| | reference and mutant amino acids separated by ‘/’; ‘X’ means |
| | any amino acid and ‘*’ means stop codon. |
| Codons | the alternative codons with the variant base in upper case |

### Example 3: Response from Pembrolizumab Treatment by Status of SETD2 Gene

The response rate to pembrolizumab by mutation status of SETD2 was summarized in ccRCC (Cohort A) and nccRCC (Cohort B) respectively. “Responders” included all patients whose best confirmed response is complete response (CR) or partial response (PR) by RECIST; “Non-Responders” included the remainder of the patients.

TABLE 5
Response rate to pembrolizumab by mutation
status of SETD2 in ccRCC and nccRCC

| | | |
|---|---|---|
| Non- | | |
| Responders | Responders | Response Rate (95% CI) |

ccRCC (N = 76)

| | | | |
|---|---|---|---|
| SETD2 Wild type | 44 | 14 | 24% (14%, 37%) |
| SETD2 Mutant | 10 | 8 | 44% (22%, 69%) |

nccRCC (N = 110)

| | | | |
|---|---|---|---|
| SETD2 Wild type | 70 | 26 | 27% (19%, 37%) |
| SETD2 Mutant | 8 | 6 | 43% (18%, 71%) |

The data suggested a trend of improved clinical response in SETD2 mutant group than SETD2 wild type group in both ccRCC and nccRCC. Though the prevalence of mutations of 5 SETD2 are different between ccRCC and nccRCC, the observed response rate by mutation status of SETD2 seems consistent across ccRCC and nccRCC.

The response rate to pembrolizumab by mutation status of SETD2 was summarized by histology in nccRCC.

TABLE 6
Response rate to pembrolizumab by mutation
status of SETD2 by histology in nccRCC

| | | |
|---|---|---|
| Non- | | |
| Responders | Responders | Response Rate (95% CI) |

Chromophobe (N = 12)

| | | | |
|---|---|---|---|
| SETD2 Wild type | 10 | 1 | 9% (0%, 41%) |
| SETD2 Mutant | 0 | 1 | 100% (3%, 100%) |

Papillary (N = 80)

| | | | |
|---|---|---|---|
| SETD2 Wild type | 49 | 20 | 29% (19%, 41%) |
| SETD2 Mutant | 7 | 4 | 36% (11%, 69%) |

Unclassified (N = 18)

| | | | |
|---|---|---|---|
| SETD2 Wild type | 11 | 5 | 31% (11%, 59%) |
| SETD2 Mutant | 1 | 1 | 50% (1%, 99%) |

The higher response rate in SETD2 mutant group compared to SETD2 wild type group was observed across all the three histologies in nccRCC. In contrast, mutations in genes encoding VHL, PBRM1, or BAP1, which are in the same cluster as SETD2 in the small arm of chromosome 3 (chr3p), were not correlated with higher response to pembrolizumab therapy. Table 7 below provides the data for mutations in these genes, and response to pembrolizumab therapy.

TABLE 7
Mutation data and response to pembrolizumab

| | |
|---|---|
| n | ORR, % (95% CI) |

| | | | | |
|---|---|---|---|---|
| Gene | Mutant | Wide type | Mutant | Wide type |

ccRCC (N = 76)

| | | | | |
|---|---|---|---|---|
| PBRM1 | 33 | 43 | 30 (16, 9) | 28 (15, 44) |
| VHL | 56 | 20 | 30 (19, 44) | 25 (9, 49) |
| BAP1 | 14 | 62 | 14 (2, 43) | 32 (21, 45) |

nccRCC (N = 110)

| | | | | |
|---|---|---|---|---|
| PBRM1 | 6 | 104 | 33 (4, 78) | 29 (20, 39) |
| VHL | 12 | 98 | 42 (15, 72) | 28 (19, 37) |
| BAP1 | 12 | 98 | 25 (5, 57) | 30 (21, 40) |

All references cited herein are incorporated by reference to the same extent as if each individual publication, database entry (e.g. Genbank sequences or GeneID entries), patent application, or patent, was specifically and individually indicated to be incorporated by reference′ each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method of treating a human patient with cancer which comprises:
(a) determining if a sample collected from the patient has reduced activity or amount of a SET domain containing 2 (SETD2) biomarker, and
(b) treating the patient with reduced activity or amount of the SETD2 biomarker with a treatment regimen that comprises administration of a therapeutically effective amount of a programmed death 1 receptor (PD-1) antagonist.

2. The method of claim 1, wherein the sample is a cancerous sample.

3. The method of claim 1, wherein the reduced activity or amount is reduced copy number of SETD2 DNA, reduced amount of SETD2 protein, reduced methylation level of the SETD2 protein substrate lysine-36 of histone H3, or reduced levels of SETD2 mRNA, as compared to a non-cancerous sample from the patient.

4. A method of treating a human patient with cancer which comprises:
(a) determining if a sample collected from the patient has a somatic mutation in a SET domain containing 2 (SETD2) nucleic acid, and
(b) treating the patient with a treatment regimen that comprises a programmed death 1 receptor (PD-1) antagonist if the somatic mutation is present.

5. The method of claim 4, wherein the somatic mutation is identified in a cancerous sample of the patient.

6. The method of claim 4, wherein the somatic mutation is a loss-of-function mutation.

7. The method of claim 4, wherein the somatic mutation is a loss-of-function nonsynonymous mutation.

8. The method of claim 4, wherein the sample has a reduced amount of SETD2 protein, reduced methylation level of the SETD2 protein substrate lysine-36 of histone H3, or reduced levels of SETD2 mRNA as compared to a non-cancerous sample from the patient.

9. The method of claim 3, wherein the amount of SETD2 protein is measured by an immunohistochemistry (IHC) assay with an anti-SETD2 antibody.

10. The method of claim 3, wherein the methylation level of its substrate lysine-36 of histone H3 is detected by an antibody to H3K36me3.

11. The method of claim 1, wherein the PD-1 antagonist is a monoclonal antibody, or an antigen binding fragment thereof.

12. The method of claim 11, wherein the PD-1 antagonist, or antigen binding fragment thereof, specifically binds to human PD-1 and blocks the binding of human PD-L1 to human PD-1.

13. The method of claim 12, wherein the PD-1 antagonist also blocks binding of human PD-L2 to human PD-1.

14. The method of claim 1, wherein the PD-1 antagonist is an antibody, or antigen binding fragment thereof, that comprises: (a) a light chain variable region comprising light chain CDR1, CDR2 and CDR3 of SEQ ID NOs: 1, 2 and 3, respectively and (b) a heavy chain variable region comprising heavy chain CDR1, CDR2 and CDR3 of SEQ ID NOs: 6, 7 and 8, respectively.

15. The method of claim 1, wherein the PD-1 antagonist is an anti-PD-1 antibody that comprises a heavy chain and a light chain, and wherein the heavy chain comprises a heavy chain variable region comprising SEQ ID NO:9 and the light chain comprises a light chain variable region comprising SEQ ID NO: 4.

16. The method of claim 1, wherein the PD-1 antagonist is an anti-PD-1 antibody that comprises two heavy chains and two light chains, and wherein the each heavy chain comprises SEQ ID NO: 10 and the each light chain comprises SEQ ID NO:5.

17. The method of claim 1, wherein the PD-1 antagonist is pembrolizumab.

18. The method of claim 17, wherein the pembrolizumab is administered at 200 mg every three weeks, or 400 mg every six weeks intravenously.

19. The method of claim 1, wherein the PD-1 antagonist is a pembrolizumab variant.

20. The method of claim 1, wherein the PD-1 antagonist is nivolumab, atezolizumab, durvalumab, cemiplimab, or avelumab.

21. The method of claim 1, wherein the patient has not been previously treated with anti-PD-1 or anti-PD-L1 therapy.

22. The method of claim 1, wherein the patient is treated with anti-PD-1 or anti-PD-L1 therapy after surgery.

23. The method of claim 1, wherein the cancer is renal cell carcinoma.

24. The method of claim 1, wherein the cancer is clear cell renal cell carcinoma.

25. The method of claim 1, wherein the cancer is non-clear cell renal cell carcinoma.

26. The method of claim 1, wherein the cancer is bladder cancer.

27. (canceled)

28. A drug product which comprises a pharmaceutical composition and prescribing information, wherein the pharmaceutical composition comprises a PD-1 antagonist and at least one pharmaceutically acceptable excipients and the prescribing information states that the pharmaceutical composition is indicated for use in a patient who has reduced activity or amount of a SETD2 biomarker.

29. The method of claim 1, wherein the cancer is melanoma, non-small cell lung cancer, head and neck squamous cell cancer, classical Hodgkin lymphoma, primary mediastinal large B-cell lymphoma, urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, or triple negative breast cancer.


SEQUENCE LISTING (12 sequences)

SEQ ID NO: 1 - protein, 15 aa, Artificial sequence
  Pembrolizumab-Light chain CDR1
RASKGVSTSGYSYLH

SEQ ID NO: 2 - protein, 7 aa, Artificial Sequence
  Pembrolizumab-Light chain CDR2
LASYLES

SEQ ID NO: 3 - protein, 9 aa, Artificial Sequence
  Pembrolizumab-Light chain CDR3
QHSRDLPLT

SEQ ID NO: 4 - protein, 111 aa, Artificial Sequence [sequence omitted]

SEQ ID NO: 5 - protein, 218 aa, Artificial Sequence
  Pembrolizumab-Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Pembrolizumab-Heavy chain CDR1
NYYMY

SEQ ID NO: 7 - protein, 17 aa, Artificial sequence
  Pembrolizumab-Heavy chain CDR2
GINPSNGGTNFNEKFKN

SEQ ID NO: 8 - protein, 11 aa, Artificial sequence
  Pembrolizumab-Heavy chain CDR3
RDYRFDMGFDY

SEQ ID NO: 9 - protein, 120 aa, Artificial sequence [sequence omitted]

SEQ ID NO: 10 - protein, 447 aa, Artificial sequence
  Pembrolizumab-Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK